(19) United States
(12) Patent Application Publication
(10) Pub. No.: US 2009/0247860 A1
(43) Pub. Date: Oct. 1, 2009

(54) SPECTRAL BIOMARKER AND ALGORITHM FOR THE IDENTIFICATION AND DETECTION OF NEURAL STEM AND PROGENITOR CELLS AND THEIR USE IN STUDYING MAMMALIAN BRAINS

(75) Inventors: Petar M. Djuric, Setauket, NY (US); Louis N. Manganas, New Hide Rock, NY (US); Grigori N. Enikolopov, Cold Spring Harbor, NY (US); Mirjana Maletic-Savatic, Houston, TX (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: RESEARCH FOUNDATION OF STATE UNIVERSITY OF NEW YORK, Albany, NY (US); COLD SPRING HARBOR LABORATORY, Cold Spring Harbor, NY (US)

(21) Appl. No.: 12/259,177

(22) Filed: Oct. 27, 2008

Related U.S. Application Data
(60) Provisional application No. 60/982,585, filed on Oct. 25, 2007.

(51) Int. Cl. A61B 5/055; G06F 19/00

(52) U.S. Cl. 600/420; 702/19

(57) ABSTRACT

The disclosure provides a biomarker and algorithm for identifying and detecting neural stem and progenitor cells and their use in studying mammalian brains. The disclosure further provides magnetic resonance spectroscopy methods and an image enhancing algorithm for the study of the proliferation of these cells and the associated neurogenesis in the live mammalian brain.

### CROSS-REFERENCES TO RELATED APPLICATIONS

The present application claims priority to U.S. Provisional Application Ser. No. 60/982,585 filed on Oct. 25, 2007, by Manganas et al., titled “MAGNETIC RESONANCE SPECTROSCOPY IDENTIFIES NEURAL PROGENITOR CELLS IN THE LIVE HUMAN BRAIN,” the entire disclosure of which is hereby incorporated by reference in its entirety for all purposes.

### STATEMENT AS TO RIGHTS TO INVENTIONS MADE UNDER FEDERALLY SPONSORED RESEARCH OR DEVELOPMENT

This invention was made with government support under grant R21NS05875-1 (MMS), 5K08 NS044276 (MMS) and R01-NS32764 (GE) awarded by the National Institute of Neurological Disorders and Stroke (NINDS); grant DAMD170110754 (M.M.S.) awarded by the US Army Medical Research; grant T32DK07521-16 (L.N.M.) awarded by the National Institute of Diabetes and Digestive and Kidney Diseases; grant FWP MO-065 (H.B.) awarded by the United States Department of Energy; grant CCF-0515246 awarded by National Science Foundation; and grant N00014-06-1-0012 (P.D.) awarded by the Office of Naval Research. The government has certain rights in the invention.

### BACKGROUND OF THE DISCLOSURE

The adult mammalian brain retains the ability to generate new neurons. These neurons are produced from neural stem and progenitor cells (NPC) that reside in the hippocampus and the subventricular zone (Lie at al., 2004; Ming and Song, 2005; Curtis et al., 2007; Goldman and Windrem, 2006). NPC possess the ability to self-renew and also to generate progeny that can give rise to mature cell types. The ability of NPC to produce neurons, astocytes, and oligodendrocytes in vitro and in vivo raises the prospect of harnessing these cells to repair nerve tissue damaged or lost to neurological disease or trauma (.Lie at al., 2004; Ming and Song, 2005; Goldman and Windrem, 2006). The realization of the curative potential of NPC would benefit from the development of methods that would enable their identification and tracking both in vitro and in vivo. Currently, Positron Emission Tomography (PET), Single Photon Computed Tomography (SPECT) scanning, and Magnetic Resonance Imaging (MRI) are being examined toward this goal (Cicchetti et al., 2007; Chin et al., 2003; Arbab et al., 2006). These technologies, however, require NPC to be preloaded ex vivo with radiolabeled agents or superparamagnetic iron oxide-based derivatives and therefore, are not applicable for the detection of endogenous NPC in the human brain. Thus, what is needed are new methods for studying neural stem and NPC in the mammalian brain.

### BRIEF SUMMARY OF THE DISCLOSURE

The disclosure provides a spectral biomarker and algorithm and methods for the identifying, detecting and quantifying neural stem and/or progenitor cells (NPC) for studying mammalian brains.

In one aspect the disclosure provides a Nuclear Magnetic Resonance (¹-H-NMR) and/or Magnetic Resonance Spectroscopy (¹H-MRS) spectral biomarker appearing as a peak in the ¹H-NMR and/or ¹H-MRS spectra in the area of approximately 1.28 (+/−0.02) parts per million (ppm) after water removal.

In another aspect the disclosure provides a Nuclear Magnetic Resonance (¹-H-NMR) and/or Magnetic Resonance Spectroscopy (¹H-MRS) spectral biomarker appearing as a peak in the ¹H-NMR and/or ¹H-MRS spectra in the area of approximately 1.28 (+/−0.02) ppm after water removal, wherein the biomarker has higher concentrations in the hippocampus and/or subventricular zone compared to other regions of a normal mammalian brain.

In another aspect the disclosure provides a Nuclear Magnetic Resonance (¹-H-NMR) and/or Magnetic Resonance Spectroscopy (¹H-MRS) spectral biomarker appearing as a peak in the ¹H-NMR and/or ¹H-MRS spectra in the area of approximately 1.28 (+/−0.02) ppm after water removal, wherein the biomarker comprises a mixture of lipids and/or peptides.

In another aspect the disclosure provides a Nuclear Magnetic Resonance (¹-H-NMR) and/or Magnetic Resonance Spectroscopy (¹H-MRS) spectral biomarker appearing as a peak in the ¹H-NMR and/or ¹H-MRS spectra in the area of approximately 1.28 ppm (+/−0.02) ppm after water removal, wherein the biomarker comprises a mixture of lipids wherein the mixture of lipids comprises saturated fatty acid (SFA) and/or monounsaturated fatty acids (MUFA).

In another aspect the disclosure provides methods for detecting neural stem and/or progenitor cells by a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells.

In another aspect the disclosure provides methods for detecting neural stem and/or progenitor cells by a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells and wherein the neural stem and/or progenitor cells are detected in vivo.

In another aspect the disclosure provides methods for detecting neural stem and/or progenitor cells by a) preparing the sample of tissue or cells for Nuclear Magnetic Resonance (¹-H-NMR) and/or Magnetic Resonance Spectroscopy (¹H-MRS) analysis; b) scanning a sample of tissue or cells using Nuclear Magnetic Resonance (¹-H-NMR) and/or Magnetic Resonance Spectroscopy (¹H-MRS); and c) detecting a spectral biomarker in the area of approximately 1.28 (+/−0.02) ppm after water removal, wherein the biomarker indicates the presence of neural stem and/or progenitor cells.

In another aspect the disclosure provides methods for detecting neural stem and/or progenitor cells by a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the intensity (area under the curve) of the peak of the biomarker in the area of approximately 1.28 ppm is used to quantify the number of neural stem and/or progenitor cells within the sample.

In another aspect the disclosure provides methods for detecting neural stem and/or progenitor cells by a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the sample of tissue or cells is in a selected area within a mammalian brain.

In another aspect the disclosure provides methods for detecting neural stem and/or progenitor cells by a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the sample of tissue or cells is in a selected area within a mammalian brain wherein the selected area is the hippocampus and/or the subventricular zone and/or the cortex of the mammalian brain.

In another aspect the disclosure provides methods for detecting neural stem and/or progenitor cells by a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the ¹H-MRS spectrum is obtained using a MRI scanner.

In another aspect the disclosure provides methods for detecting neural stem and/or progenitor cells by a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the MRI scanner is a 9.4T Biospec Avance 94/92 as scanner.

In another aspect the disclosure provides methods for detecting neural stem and/or progenitor cells by a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the MRI scanner is a 3T MRI scanner.

In another aspect the disclosure provides methods for monitoring transplanted neural stem and/or progenitor cells in a selected area of a mammalian brain by: a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells.

In another aspect the disclosure provides methods for evaluating the efficacy of a therapeutic intervention as measured by an increases or decrease in the number of neural stem and/or progenitor cells in a selected area of a mammalian brain by: a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells.

In another aspect the disclosure provides methods for evaluating the efficacy of a therapeutic intervention as measured by an increases or decrease in the number of neural stem and/or progenitor cells in a selected area of a mammalian brain by: a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the therapeutic intervention is for a nervous system disorder related to cellular degeneration, a psychiatric condition, cellular trauma and/or injury, or another neurologically related condition in a mammalian subject or patient.

In another aspect the disclosure provides methods for evaluating the efficacy of a therapeutic intervention as measured by an increases or decrease in the number of neural stem and/or progenitor cells in a selected area of a mammalian brain by: a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the therapeutic intervention includes but is not limited to a nervous system disorder related to cellular degeneration in a mammalian subject or patient wherein the nervous system disorder related to cellular degeneration includes but is not limited to a neurodegenerative disorder, a neural stem cell disorder, a neural progenitor cell disorder, a degenerative disease of the retina, an ischemic disorder, demyelinative, inflammatory, degenerative, metabolic, vascular, epileptogenic, neoplastic, related to premature birth, increased intracranial pressure, dementia or combinations thereof.

In another aspect the disclosure provides methods for evaluating the efficacy of a therapeutic intervention as measured by an increases or decrease in the number of neural stem and/or progenitor cells in a selected area of a mammalian brain by: a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the therapeutic intervention is for a nervous system disorder related to a psychiatric condition in a mammalian subject or patient wherein the nervous system disorder related to a psychiatric condition includes but is not limited to a neuropsychiatric disorder, an affective disorder, depression, hypomania, panic attacks, anxiety, excessive elation, bipolar depression, bipolar disorder (manic-depression), seasonal mood (or affective) disorder, schizophrenia and other psychoses, lissencephaly syndrome, anxiety syndromes, anxiety disorders, phobias, stress and related syndromes, cognitive function disorders, aggression, drug and alcohol abuse, obsessive compulsive behavior syndromes, borderline personality disorder, non-senile dementia, post-pain depression, post-partum depression, cerebral palsy, post-traumatic distress disorder (PTSD), or combinations thereof.

In another aspect the disclosure provides methods for evaluating the efficacy of a therapeutic intervention as measured by an increases or decrease in the number of neural stem and/or progenitor cells in a selected area of a mammalian brain by: a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the therapeutic intervention is for cellular trauma and/or injury in a mammalian subject or patient wherein the nervous system disorder related to cellular trauma and/or injury includes but is not limited to neurological traumas and injuries, surgery related trauma and/or injury, retinal injury and trauma, injury related to epilepsy, spinal cord injury, brain injury, brain surgery, trauma related brain injury, trauma related to spinal cord injury, brain injury related to cancer treatment, spinal cord injury related to cancer treatment, brain injury related to infection, brain injury related to inflammation, spinal cord injury related to infection, spinal cord injury related to inflammation, brain injury related to environmental toxin, spinal cord injury related to environmental toxin, or combinations thereof.

In another aspect the disclosure provides methods for evaluating the efficacy of a therapeutic intervention as measured by an increases or decrease in the number of neural stem and/or progenitor cells in a selected area of a mammalian brain by: a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the therapeutic intervention is for a nervous system disorder related to a neurologically related condition in a mammalian subject or patient wherein the neurologically related condition includes but is not limited to learning disorders, memory disorders, autism, attention deficit disorders, narcolepsy, sleep disorders, cognitive disorders, epilepsy, temporal lobe epilepsy, or combinations thereof.

In another aspect the disclosure provides methods for evaluating the efficacy of a therapeutic intervention as measured by an increases or decrease in the number of neural stem and/or progenitor cells in a selected area of a mammalian brain by: a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the therapeutic intervention is for a nervous system disorder related to a psychiatric condition in a mammalian subject or patient wherein the nervous system disorder related to a psychiatric condition is depression.

In another aspect the disclosure provides methods for evaluating the efficacy of a therapeutic intervention as measured by an increases or decrease in the number of neural stem and/or progenitor cells in a selected area of a mammalian brain by: a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the therapeutic intervention is for a nervous system disorder related to a psychiatric condition in a mammalian subject or patient wherein the nervous system disorder related to a psychiatric condition is PTSD.

In another aspect the disclosure provides methods for evaluating the efficacy of a therapeutic intervention as measured by an increases or decrease in the number of neural stem and/or progenitor cells in a selected area of a mammalian brain by: a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the increase or decrease in the number of neural stem and/or progenitor cells is affected through the therapeutic intervention.

In another aspect the disclosure provides methods for evaluating the efficacy of a therapeutic intervention as measured by an increases or decrease in the number of neural stem and/or progenitor cells in a selected area of a mammalian brain by: a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the increase or decrease in the number of neural stem and/or progenitor cells is affected through the therapeutic intervention wherein the increase in the number of neural stem and/or progenitor cells is correlated with increased neurogenesis, and wherein the decrease in the number of neural stem and/or progenitor cells is correlated with decreased neurogenesis.

In another aspect the disclosure provides methods for evaluating the efficacy of a therapeutic intervention as measured by an increases or decrease in the number of neural stem and/or progenitor cells in a selected area of a mammalian brain by: a) scanning a sample of tissue or cells using Nuclear Magnetic Resonance (¹-H-NMR) and/or Magnetic Resonance Spectroscopy (¹H-MRS); and b) detecting a spectral biomarker in the area of approximately 1.28 (+/−0.02) ppm after water removal, wherein the biomarker indicates the presence of neural stem and/or progenitor cells wherein the mammalian brain is a human brain.

In another aspect the disclosure provides signal-processing algorithms for isolating a ¹H-MRS signal from background noise.

In another aspect the disclosure provides signal-processing algorithms for isolating a ¹H-MRS signal from background noise wherein the algorithm is utilized to enhance the ¹H-MRS spectra.

In another aspect of the disclosure, a system for identifying the presence of a biomarker from MRS data includes: an input configured to receive the MRS data; a memory configured to store the MRS data; and a processor configured to operate on the stored MRS data to: reduce an influence of water data in the MRS data; determine that a signal-to-noise ratio of the MRS data with reduced water data influence is within a desired range; calibrate the MRS data with reduced water data influence; reduce influences of signals in close proximity, as a function of parts per million (ppm), to a ppm value of interest; and determine whether a signal exists approximately at the ppm value of interest.

Embodiments of such systems may include one or more of the following features. The processor, in order to determine whether a signal of a strength above a threshold exists at the ppm value of interest, is configured to: estimate sets of signal components using models of different orders; construct spectra from the estimated sets of signal components; and determine if the constructed spectra are within an acceptable difference relative to a calculated spectrum determined using a Fourier transform of the MRS data. The processor, in order to determine whether a signal of a strength above a threshold exists at the ppm value of interest, is further configured to: determine whether a sufficiently strong signal, of a strength above a threshold, exists in a vicinity of the ppm of interest for each of the estimates for which the constructed spectra was within the acceptable difference; and determine whether a damping factor for each sufficiently strong signal is in an acceptable range. The processor, in order to determine whether a signal of a strength above a threshold exists at the ppm value of interest, is further configured to compare the sufficiently strong signals for consistency. The processor, in order to determine whether a signal of a strength above a threshold exists at the ppm value of interest, is further configured to combine the sufficiently strong signals into an indication of the signal that exists approximately at the ppm value of interest. The processor is configured to use the indication to estimate a quantity of cells.

In another aspect of the disclosure, a computer program product resides on a computer-readable medium and includes computer-readable instructions that will cause a computer to: reduce an influence of water data in the MRS data; determine that a signal-to-noise ratio of the MRS data with reduced water data influence is within a desired range; calibrate the MRS data with reduced water data influence; reduce influences of signals in close proximity, as a function of parts per million (ppm), to a ppm value of interest; and determine whether a signal exists approximately at the ppm value of interest.

In another aspect of the disclosure, a computer program product resides on a computer-readable medium and includes computer-readable instructions that will cause a computer to: perform analysis of metrics of parametric and non-parametric spectra of MRS data; and determine from the analysis whether estimates of signals within the MRS data are acceptable.

In another aspect of the disclosure, a computer program product resides on a computer-readable medium and includes computer-readable instructions that will cause a computer to: perform analysis of parametric and non-parametric spectra of MRS data; and determine from the analysis whether a biomarker is indicated by the MRS data. Embodiments of such computer program products may include instructions that will further cause the computer to determine a set of acceptable spectra and determine from the set of acceptable spectra whether the biomarker is indicated by the MRS data.

In another aspect of the disclosure, a computer program product resides on a computer-readable medium and includes computer-readable instructions that will cause a computer to: estimate signal parameters, indicated by MRS data, for a biomarker of interest; and estimate a quantity of tissue cells contributing to a signal strength of the biomarker.

### BRIEF DESCRIPTION OF THE DRAWINGS

FIG. 1A shows the spectral profiles of cultured neural cell types: Neural Stem and Progenitor Cells (NPC), Neurons (N), Oligodendrocytes (O) and Astrocytes (A). Dotted lines outline the 1.28 ppm NPC peak. N-acetyl aspartate (NAA), a biomarker for neurons, is represented by the peak at 2.02 ppm; and choline (Cho), a biomarker for astrocytes, is represented by the peak at 3.23 ppm. Arrowheads denote lactate doublets at 1.33ppm.

FIG. 1B shows the quantification of the 1.28 ppm biomarker (top), NAA (middle) and Cho (bottom) for each neural cell type (2.5×10⁵ cells each; N=3).

FIG. 1C shows the correlation of the 1.28 ppm biomarker with the number of NPC (N=3 per group).

FIG. 1D shows the quantification of the 1.28 ppm biomarker in proliferating cells: NPC, embryonic stem cells (ESC), hair follicle-derived progenitor cells (SPC), oligodendrocyte progenitor cells (OPC), macrophages (MΦ), T lymphocytes (TC), and microglia (MG) at 1×10⁶ cells each (N=3).

FIG. 2 shows FACS analysis and 1.28 ppm biomarker content of nestin-GTP cells obtained from neurospheres cultured from nestin-GFP transgenic C57bL/6 mice.

FIG. 3(A-G) shows the analysis of the specificity and molecular composition of the NPC biomarker using ¹H-NMR. FIG. 3A shows the quantification of NPC, neuronal (NAA) and glial (Cho) biomarkers during in vitro differentiation, at 0, 1 and 5 days after neurosphere plating (1×10⁶ cells per time point, N=3). FIG. 3B shows the quantification of NPC, neuronal (NAA) and glial (Cho) biomarkers in whole brain homogenates at embryonic day 12 (E12) and postnatal day 30 (P30) (1×10⁶ cells per time point, N=3). FIG. 3C shows the quantification of the NPC biomarker in dissociated adult mouse cortex (CTX) and hippocampus (HIPP) (1×10⁶ cells per time point, N=3). FIG. 3D shows increases in both BrdU immunoreactive cells (N=3; p<0.01) and the 1.28 ppm biomarker (N=3; p<0.01) as a result of electroconvulsive shock (ECS). FIG. 3E shows the 1.28 biomarker decreasing and the Cho biomarker increasing in response to the blockade of fatty acid synthesis with cerulenin (CRL) (N=3; p<0.001). FIG. 3F shows that SFA and MUFA are more abundant in NPC than in astrocytes (N=1). FIG. 3G shows the 1.28 ppm biomarker belongs to the chloroform (CCl₃D) and not the methanol (MeOD) fraction. The 1.28 ppm biomarker overlaps with saturated (SFA) and monounsaturated fatty acids (MUFA), rather than polyunsaturated fatty acids (PUFA).

FIG. 4(A-E) shows the in vivo identification of NPC in the rat brain, using micro MRI spectroscopy. FIG. 4A shows the imaging of endogenous NPC. Voxels are placed along the hippocampus (HIPP) and in the cortex (CTX). In the hippocampus, the 1.28 ppm biomarker (red) is evident when SVD-based signal processing is performed (colored peaks) but not when Fourier transform is done (insets). In the cortex, the 1.28 ppm biomarker is not detected by either data analysis. Colored asterisks and peaks correlate. Bar graphs show absolute (top) and relative (bottom) quantification of the 1.28 ppm biomarker (N=4, p<0.05). FIG. 4B shows the imaging of transplanted NPC. Voxels are placed in the area of the NPC transplant (NT; 5×10⁶ NPC in 5 μL of saline) and saline injection (ST; 5 μL). In the NT site, the 1.28 ppm biomarker (red) is observed with both Fourier transform and SVD-based signal processing. In the ST site, no significant 1.28 ppm signal is observed. Bar graphs show absolute (top) and relative (bottom) quantification of the 1.28 ppm biomarker (N=5; p<0.05). FIG. 4(C-E) shows the imaging of endogenous NPC after ECS. Voxels are placed along the hippocampus in control (ESC−) and ECS-treated (ECS+) adult rats (C). Quantification of the 1.28 biomarker (C; N=4, p<0.05) and the number of BrdU immunoreactive cells in the dentate gyrus of the same animal (D; N=4, p<0.01) indicates a linear correlation (E).

FIG. 5(A-C) shows the in vivo identification of NPC in the human hippocampus using ¹H-MRI spectroscopy. FIG. 5A shows the location of voxels placed along the hippocampus and in the cortex. In the hippocampus, the 1.28 ppm biomarker (red) is evident when SVD-based signal processing is performed but not when Fourier transform is done. In the cortex, the 1.28 ppm biomarker is not detected by either analysis. Colored asterisks and colored peaks correlate. Bar graphs show absolute (top) and relative (bottom) quantification of the 1.28 ppm biomarker (CTX=cortex, LH=left hippocampus, RH=right hippocampus: N=5, p<0.0 1 and p<0.05 respectively). FIG. 5B shows the quantification of the 1.28 ppm biomarker in the adult human hippocampus over time (N=4,p=0.747). Same subjects were imaged 90 days apart. FIG. 5C shows the quantification of the 1.28 ppm biomarker in the human hippocampus during development: pre-adolescent, adolescent and adult age groups (N=3 per group; p<0.001).

FIG. 6 is a simplified diagram of an MRS system for in vivo measurements.

FIG. 7 is a block diagram of a computer shown in FIG. 6.

FIG. 8-9 are a block flow diagram of a process of processing MRS data acquired by the system shown in FIG. 6.

### DETAILED DESCRIPTION OF THE DISCLOSURE

The disclosure provides a unique biomarker and algorithm, that has been identified through ¹H-NMR spectral analysis, and methods for identifying and detecting neural stem and/or progenitor cells (NPC) using this biomarker and algorithm for studying the mammalian brain. The image enhancing algorithm provided allows for the isolation and enhancement of this biomarker from background noise in the ¹H-MRS spectrum. The biomarker and the enhancing algorithm may be used to track and analyze endogenous and/or exogenous NPC, to monitor neurogenesis in a wide range of neurological and psychiatric disorders, and to evaluate the efficacy of therapeutic interventions.

Proton Nuclear Magnetic Resonance Spectroscopy (¹-H-NMR) is useful for the in vitro detection of low quantities of known metabolites and for the identification of unknown compounds present in body fluids or tissues. ¹H-NMR can also identify metabolites that are specific for neurons (e.g., N-actyl aspartate, NAA) or glia (e.g., choline, Cho, and myoinositol, ml), which may be used as reliable biomarkers of the corresponding cell types in isolated tissue samples. However, ¹H-NMR cannot be used to analyze metabolites in live organisms. Instead, its correlate, ¹H-MRS, is used to provide information about the metabolic status of a tissue in vivo. These techniques complement each other when physiological or pathological states are investigated. Thus, the embodiments described herein also provide NPC-specific metabolites that are identified using ¹H-NMR and information about these metabolites may be used for detecting NPC in the live brain using ¹H-MRS.

The disclosure also provides a signal processing algorithm that isolates the 1.28 ppm peak from the ¹H-MRS spectrum. This 1.28 ppm biomarker may be used for the in vivo analysis of the living mammalian brain. The correlation of the 1.28 ppm biomarker with NPC and neurogenesis observed in vitro utilizing ¹H-NMR was substantiated with the in vivo analysis of the mammalian brain using ¹H-MRS with the signal enhancing algorithm. This correlation is demonstrated in both rat and man.

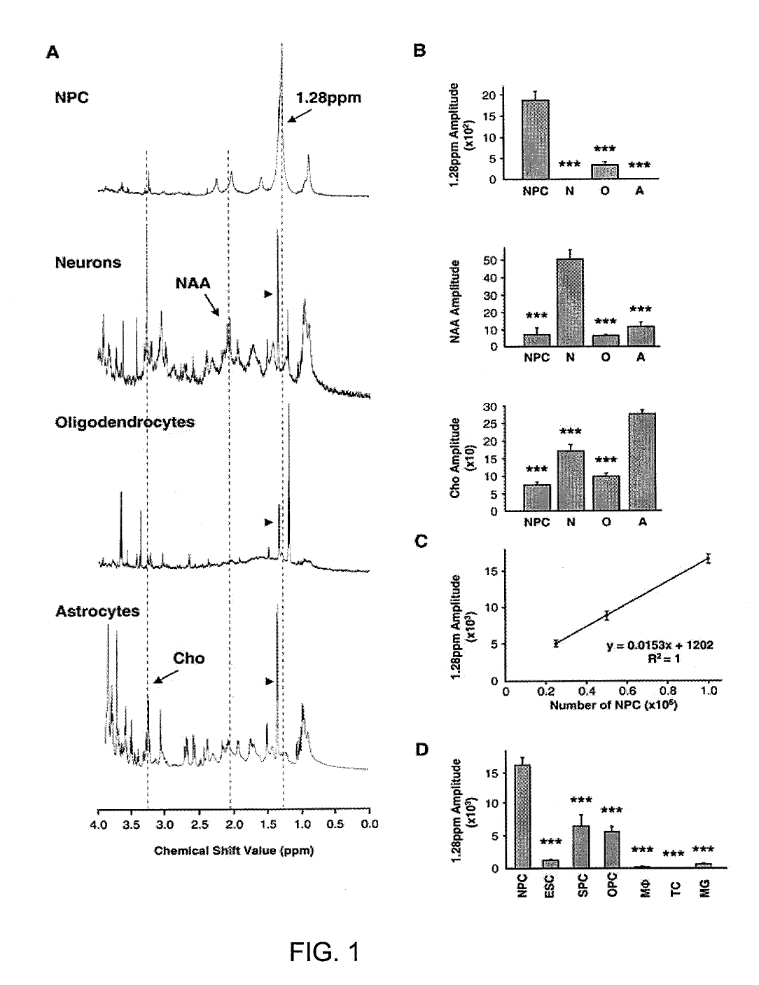

The ¹H-NMR spectra of NPC from embryonic mouse brain cultivated as neurospheres in vitro demonstrates a unique profile, including a prominent peak at the frequency of 1.28 (+/−0.02) ppm after removal of water, which is not observed in other cell types. It has been found that this biomarker has higher concentrations in cells isolated from the neurogenic regions of the brain known to be enriched with NPC, such as the hippocampus and/or subventricular zone where continuous neurogenesis takes place, but is significantly lower in other regions of a normal mammalian brain, for example, cells isolated from the cortex where neurogenesis is not detectable. The presence of this biomarker is also significantly greater in cultured NPC than in cultured neurons, oligodendrocytes or astrocytes (FIG. 1A-B). The presence of this biomarker in cultured NPC (based on the respective ¹H-NMR spectroscopic profiles) is also significantly greater than that observed in other cell types such as embryonic stem cells (ESC), cells of the hair follicle-derived sphere cultures (SPC), oligodendrocyte progenitor cells (OPC) as well as cells that may be present in the brain such as macrophages, T lymphocytes and microglia (FIG. 1D). The 1.28 ppm biomarker also directly correlates with the number of NPC within the selected sample (FIG. 1C). This information may be used for direct quantification of NPC content based on spectral analysis.

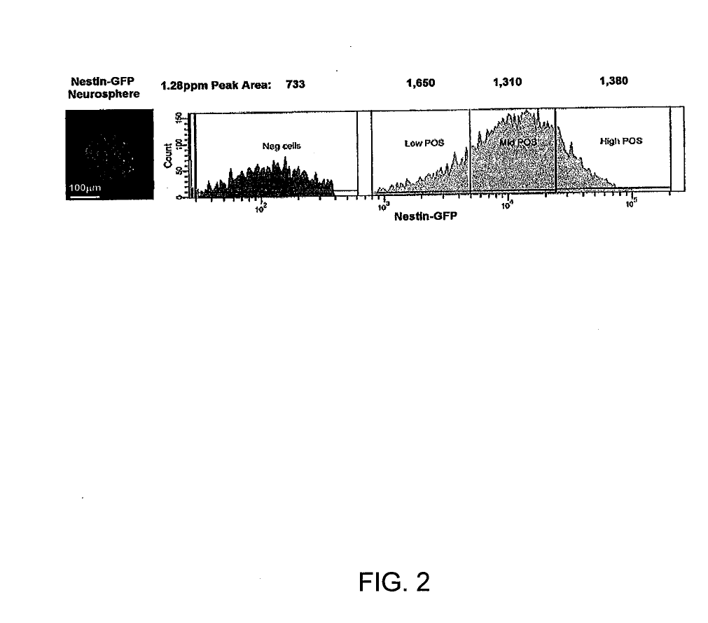

The 1.28 ppm biomarker for identifying and detecting NPC is demonstrated by experiments conducted on cultured neurospheres derived from brains of transgenic mice expressing green fluorescent protein (GFP) under control of nestin gene regulatory elements (Mignone et al., 2004). Nestin-GFP neurospheres were dissociated and the cells were sorted based on GFP expression levels using fluorescence activated cell sorting (FIG. 2). NPC-enriched GFP-expressing cell population contained higher levels of the 1.28 ppm biomarker than the GFP-negative cells. These experiments indicate that progenitor cells of different origin but each having neural potential express the 1.28 ppm biomarker. These experiments also show that among the panel of cells tested, NPC have the highest level of the 1.28 biomarker while this biomarker was absent in both post mitotic differentiated cells and cells without progenitor properties.

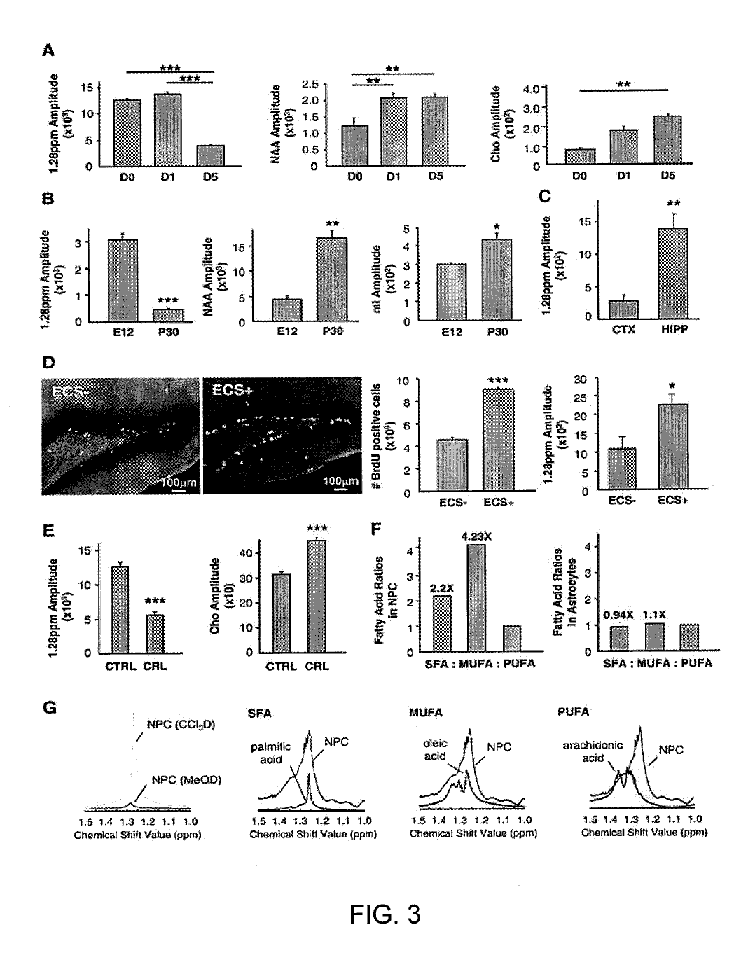

The 1.28 ppm biomarker can be correlated with the status of progenitor cells examined both in vitro and in vivo with comparison to biomarkers specific for differentiated cells. Neurospheres were cultivated under conditions that promote neuronal and astrocyte differentiation and were analyzed by ¹H-NMR. Under these conditions, it was found that the levels of the 1.28 ppm biomarker decreased, whereas the levels of the neural biomarker NAA and astrocyte biomarker Cho increased after several days of cultivation (FIG. 3A). The spectra of cells isolated from the mouse brain at embryonic day 12 (E12) when neurogenesis begins, and at postnatal day 30 (P30) when most of the cells have already differentiated were also compared. The levels of the 1.28 ppm biomarker were significantly reduced, whereas the levels of biomarkers of differentiated cells were significantly elevated in the postnatal brain as compared to the embryonic brain (FIG. 3B).

In addition, the 1.28 ppm biomarker was examined in different regions of the adult mouse brain. The ¹H-NMR spectra of cells isolated from the adult mouse hippocampus, where continuous neurogenesis takes place, and from the cortex, where neurogenesis is not detected, were compared ((Lie at al., 2004; Ming and Song, 2005; Bhardwaj, 2006). A significantly higher amount of 1.28 ppm biomarker was observed in the adult hippocampus as compared to the cortex (FIG. 3C) demonstrating that the 1.28 ppm biomarker correlates with the presence of NPC.

It has also been found that the 1.28 ppm biomarker correlates with dynamic changes in adult neurogenesis. The effect of electroconvulsive shock (ECS) on the adult mammalian hippocampus was examined. It has been shown that the adult mammalian hippocampus is sensitive to a wide range of stimuli, including ECS (van Praag et al., 1999; Kempermann et al., 1997; Encinas et al., 2006; Warner-Schmidt and Duman, 2006;Madsen et al., 2000; Perera et al., 2007). ECS was applied to adult mice and cell proliferation assessed using BrdU incorporation in the subgranular zone of the dentate gyrus and compared to 1.28 ppm biomarker levels measured using ¹H-NMR. The number of BrdU-immunoreactive cells was significantly increased in ECS-treated animals as compared to the sham-operated animals demonstrating the effectiveness of the procedure (FIG. 3D). The levels of the 1.28 ppm biomarker in the preparation of cells from the hippocampus were also significantly increased after ECS-treatment (FIG. 3D). Together, the results of cultured NPC and the developing and adult brain demonstrate that the amount of 1.28 ppm biomarker correlates with neurogenesis and demonstrates that changes neurogenesis, i.e., an increase or a decrease in the level of neurogenesis, may be analyzed using the 1.28 ppm biomarker as a valid reference for NPC.

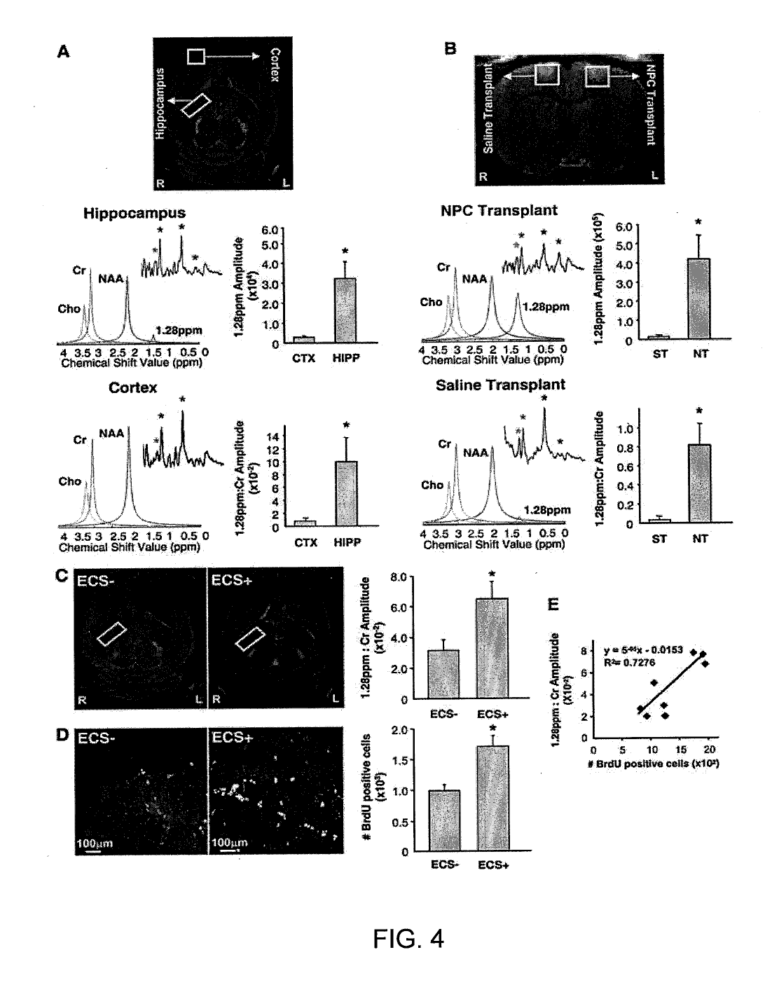

The chemical nature of the 1.28 ppm biomarker was also characterized. A specific group of resonances in the 0-2ppm range of the ¹H-NMR arises from macromolecules containing fatty acyl chains of triacylglycerides and cholesterol esters found in free floating mobile lipids in the cytoplasm and in unrestricted lipid microdomains near the plasma membrane (Sparling et al., 1989). Proton chemical shift correlation spectroscopy (COSY) of NPC demonstrates a J-coupling partner for the 1.28 ppm biomarker that resonates at 0.8 ppm, as would be expected for a fatty acid containing methyl (—CH₃) groups on the same molecule (FIG. 4; also see FIG. 1A). Further evidence that the 1.28 ppm biomarker corresponds to lipids is the decrease in the 1.28 ppm peak area when neurospheres are treated with cerulenin, an inhibitor of fatty acid synthesis (FIG. 3E).

To further demonstrate that the 1.28 ppm biomarker contains lipids, the ¹H-NMR spectra of NPC extracted with a chloroform/methanol mixture was analyzed. The 1.28 ppm biomarker was mainly present in the chloroform fraction indicating the presence of a lipid metabolite (FIG. 3G). This biomarker also overlapped with some of the specific fatty acid spectra, most closely with the spectra of saturated fatty acids (SFA), such as palmitic acid, and of monounsaturated fatty acids (MUFA), such as oleic acid (FIG. 2G [SAME]). The spectra of polyunsaturated fatty acids (PUFA), such as arachidonic acid, which resonate in the 1.3-1.4 ppm range, did not overlap (FIG. 2G [SAME]). Gas chromatography was used to separate and quantify specific fatty acids in NPC and they were compared to those found in astrocytes. SFA and MUFA were enriched relative to PUFA in NPC but not in astrocytes (FIG. 3F). Together, these results demonstrates that the 1.28 ppm biomarker is a mixture of lipids that include SFA and/or MUFA. In addition, as cerulenin does not completely abolish the 1.28 ppm signal, the molecule most likely in addition to fatty acids contains a peptide or other chemical part.

The disclosure also provides the unique 1.28 ppm biomarker described above for in vivo brain imaging. Using a 9.4T mMRI scanner, adult rat spectra of the hippocampus was obtained, where endogenous NPC reside, and the parietal cortex, where dividing NPC are undetectable (FIG. 4). Traditional Fourier transform signal processing was unable to distinguish the 1.28 ppm biomarker in the hippocampus from background noise (FIG. 4A, insets) due to a low NPC density in the adult rat hippocampus. Therefore, a more sensitive signal processing algorithm was developed in order to isolate the signal of the 1.28 ppm biomarker from the noise within the in vivo ¹H-MRS spectra. Singular value decomposition (SVD) was used, which permits improved detection at low signal-to-noise ratios and allows better resolution of signal components (modes) that are close to one another in a given frequency domain (Barkhusen et al., 1987; Cavassila et al., 1997; Stoica et al., 2003). Based on SVD signal processing, an algorithm was developed that detects the 1.28 ppm biomarker in the adult rat hippocampus in vivo (FIG. 3A, peak labeled 1.28 ppm).

Absolute quantification of the 1.28 ppm biomarker was achieved by estimating the amplitude of the 1.28 ppm peak, while relative quantification was achieved by ratiometric analysis with the creatine (Cr) peak amplitude as a denominator. Both quantification methods are established as reliable indicators of a given metabolite concentration (Dieterle et al., 2006).

Both absolute quantities of the 1.28 ppm biomarker and relative quantitations of the 1.28 ppm biomarker, based on ratiometric quantification of the spectral peaks, of the hippocampal and cortical spectra confirmed that the hippocampus was highly enriched in the 1.28 ppm biomarker compared to cortex (FIG. 4A).

The in vivo utility of the 1.28 ppm biomarker is demonstrated by the transplantation of NPC into the left cortical hemisphere of adult rat brain in which an equal volume of saline was injected into the control right hemisphere. ¹H-MRS data were obtained for both hemispheres from voxels of the same size, centered on the injected areas (FIG. 3B). Both Fourier transform (inset) and SVD-based signal processing clearly detected the 1.28 ppm biomarker in the spectra of the experimental site containing NPC (FIG. 3B). Both the absolute quantification and the ratiometric analysis demonstrated that the signal in the hemisphere with the injected NPC was more than thirty five fold greater than that in the corresponding region of the control cortical hemisphere (FIG. 3B).

To detect changes in the density of endogenous NPC in vivo, adult rats were treated with ECS. Five days after the treatment, BrdU was injected to label dividing cells and the ECS-treated and sham-operated control animals were analyzed the next day. Quantification of the 1.28 ppm:Cr peak area ratios in the hippocampus showed a significant increase of the 1.28 ppm biomarker in ECS-treated rats as compared to sham-operated controls (FIG. 3C). To validate the spectroscopic findings, the number of BrdU immunoreactive cells in the hippocampus of the same animals were quantified (FIG. 3D). A significant increase in the number of BrdU immunoreactive cells in ECS-treated rats compared to sham-operated controls demonstrated that ECS increased NPC proliferation. Moreover, in the ECS-treated animals, a correlation was found between the number of BrdU immunoreactive cells and the 1.28 ppm:Cr peak area ratio in the hippocampus of the same animal (FIG. 3E). Together, these data indicate that ¹H-MRS can be used to detect and measure changes in the density of endogenous NPC in vivo.

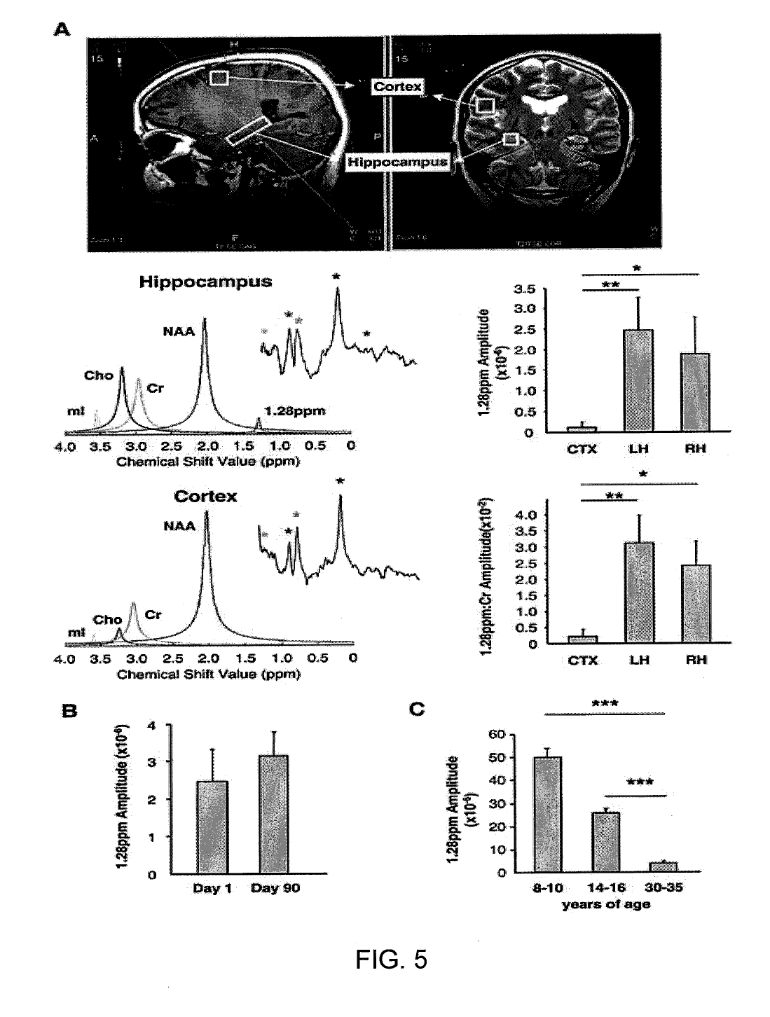

The in vivo utility of the 1.28 ppm biomarker is also demonstrated by the identification of endogenous NPC in the human brain. Brain ¹H-MRS was performed on five healthy adult subjects, using a 3T MRI scanner and SVD-based signal processing (FIG. 5A). An experimental voxel was placed along the length of the hippocampus, while a control voxel of the same volume included gray and white matter of the ipsilateral parietal cortex (FIG. 5A). The Fourier transform did not reveal the 1.28 ppm biomarker (inset) in any of the voxels (FIG. 5A). However, the SVD-based analysis (peaks) clearly detected the 1.28 ppm biomarker in the hippocampal spectra, indicating that this methodology can be used to identify endogenous NPC in the human brain (FIG. 5A). For each subject, a major difference was found between the hippocampus and the cortex using either absolute or ratiometric quantification of the 1.28 ppm biomarker, indicating that both can be applied to measure NPC density in the human hippocampus. No difference in the level of the 1.28 ppm biomarker was observed when the left and right hippocampi were compared (FIG. 5A). When the left hippocampus of the same subjects was imaged after a three-month period during which there was no major change in their daily routine, no difference was observed in the 1.28 ppm biomarker (FIG. 5B). Finally, the age-related changes were analyzed in the 1.28 ppm biomarker during human development by imaging subjects of varying ages: preadolescents, adolescents, and adults. Quantification of the 1.28 ppm biomarker revealed a decrease in the 1.28 ppm peak area (FIG. 5C), compatible with data demonstrating age-related decrease in neurogenesis in animals (Kuhn et al., 1996).

A more sensitive signal processing algorithm was also developed in order to isolate the signal of the 1.28 ppm biomarker from the noise within the in vivo ¹H-MRS spectra. A singular value decomposition (SVD) was used which permits improved detection at low signal-to-noise ratios and allows better resolution of signal components (modes) that are close to one another in a given frequency domain (Barkhusen et al., 1987; Cavassila et al., 1997; Stoica et al., 2003). Based on SVD signal processing, an algorithm was developed that detects the 1.28 ppm biomarker in the adult rat hippocampus in vivo (FIG. 4A). Absolute quantification of the 1.28 ppm biomarker was achieved by estimating the area under the 1.28 ppm peak, while relative quantification was achieved by ratiometric analysis with the creatine (Cr) peak area as a denominator. Both quantification methods are established as reliable indicators of a given metabolite concentration (Dieterle et al., 2006). A large difference was observed when the absolute quantities of the 1.28 ppm biomarker were compared between the hippocampal and cortical spectra; this was paralleled by the ratiometric quantification which confirmed that the hippocampus was highly enriched in the 1.28 ppm biomarker compared to cortex (FIG. 4A). This algorithm was also successfully used to isolate the 1.28 ppm biomarker in the human brain.

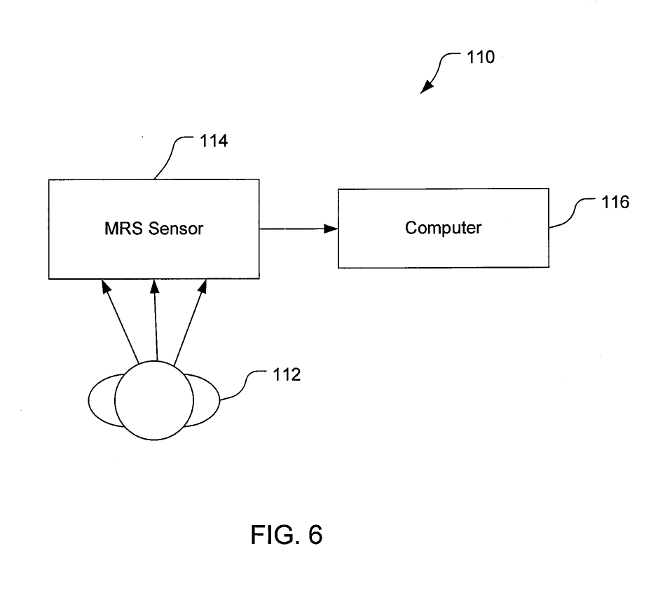

Referring to FIG. 6, an MRS system 110 includes a patient 112, an MRS sensor 114, and a computer 116. The system 110 is configured to determine biochemical information about the patient 112, specifically whether tissue of interest is present, at least in a relevant amount, in the patient 112. The MRS sensor 114 can acquire signals from chemical nuclei of biochemicals (metabolites) in the patient 112 (looking down on the patient's head in FIG. 6). The sensor conveys data regarding the acquired signals to the computer 116. Some preprocessing may occur before the data are provided to the computer 116. Further, the data may be provided to the computer 116 via a direct connection, via a network connection (e.g., a wide-area network, a local-area network, etc.), etc.

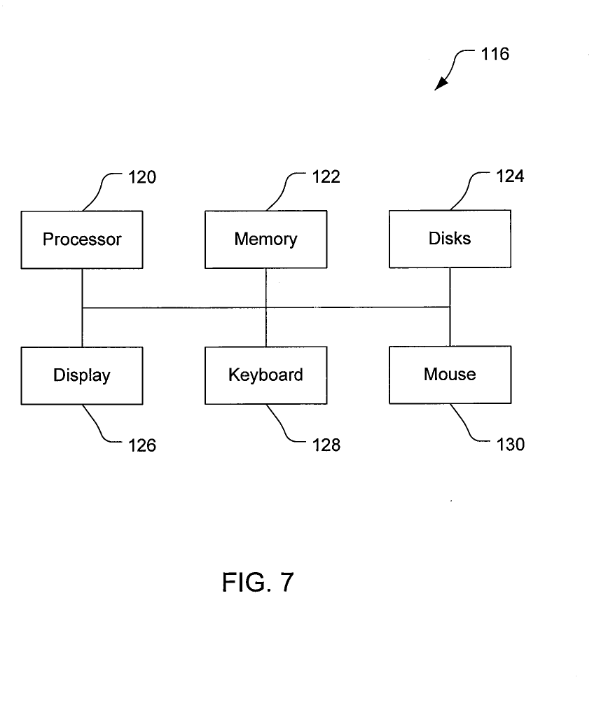

Referring also to FIG. 7, the computer 116 includes a processor 120, memory 122, disk drives 124, a display 126, a keyboard 128, and a mouse 130. The processor 120 is preferably an intelligent device, e.g., a personal computer central processing unit (CPU) such as those made by Intel® Corporation or AMD®, a microcontroller, an application specific integrated circuit (ASIC), etc. The memory 122 includes random access memory (RAM) and read-only memory (ROM). The disk drives 124 include a hard-disk drive and can include floppy-disk drives, a CD-ROM drive, and/or a zip drive. The display 128 is a cathode-ray tube (CRT), although other forms of displays are acceptable, e.g., liquid-crystal displays (LCD), TFT displays, etc. The keyboard 128 and the mouse 130 provide data input mechanisms for a user (not shown), although other input devices may be used instead of or in addition to the keyboard 128 and/or the mouse 130. The computer 116 can store, e.g., in the memory 122 and/or the disks 124, software code containing computer-readable (and preferably computer-executable) instructions for controlling the processor 120 to perform functions described here.

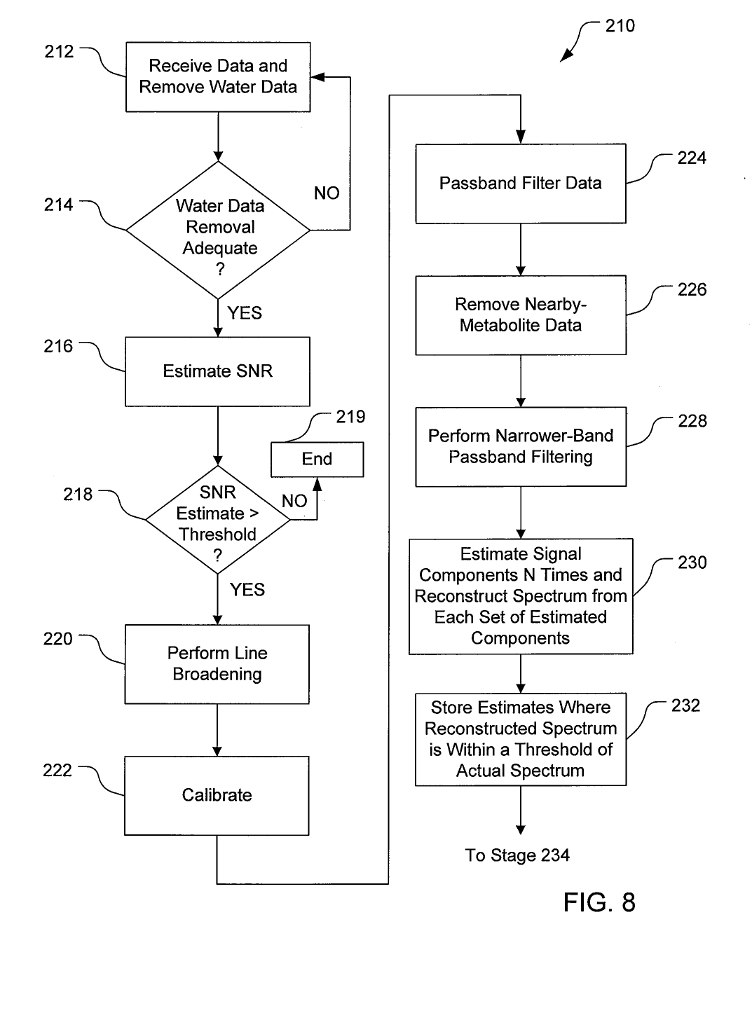

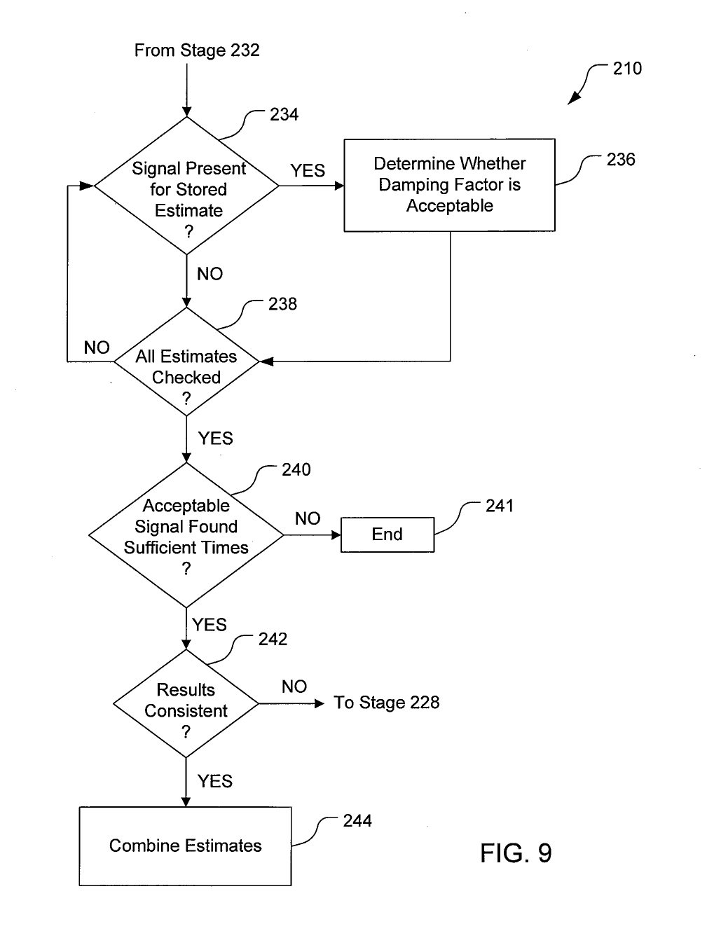

Referring also to FIG. 8-9, a process 210 of processing MRS data to determine whether a relevant biomarker is present in a relevant amount includes the stages shown. The process 210 is, however, exemplary only and not limiting. The process 210 can be altered, e.g., by having stages added, removed, or rearranged. For example, stage 218 discussed below may be omitted. Other modifications to the process 210 are possible. The process 210 can detect a signal at a particular frequency, that is approximately known in advance, in Nuclear Magnetic Resonance and/or Magnetic Resonance Spectroscopy data. Here, the process 210 is specifically designed to detect a signal that is specific for stem cells and represents their signature. The presence of the signal in the data means that there are stem cells in a scanned voxel; otherwise, it is concluded that the voxel does not contain them. In addition to determining from the data if stem cells are in the scanned tissue, the process 210 can be used for estimation of the quantity of the scanned stem cells. This estimate is based on the correlation that exists between the strength of the signal (its power) and the quantity of the stem cells. The process 210 includes removal of the water signal from the data, calibrations of the spectra, filterings, and detection and estimation procedures. An analogous procedure can be applied for detection/estimation of signals that characterize other metabolites. The process 210 is based on parametric modeling of the signals, with the signals in the data represented by mathematical functions described by only a few parameters. These functions are decaying complex sinusoids, where each sinusoid is defined by four types of parameters: amplitudes, initial phases, frequencies, and damping factors. As the biomarker is usually a signal much weaker than other signals in the data, data corresponding to other signals are removed to help reduce the possible obscuring of the biomarker by these signals. The parameters of the signals are estimated, and the signals can be removed by reconstructing them and subtracting them from the data. Other removals of unwanted signals and noise include filtering methods. With undesired signal data removed, the desired biomarker is determined, e.g., estimated.

At stage 212, MRS data are received and water data removed. While data are removed, and preferably all water-related data would be removed, less than all of the water-related data may be removed in actuality. Raw MRS data are received by the computer 116 from the sensor 114. The raw data may be pre-processed to some extent before reaching the computer 116, e.g., having been processed by an operator of the sensor 114. A fast Fourier transform (FFT) of the raw data is computed and the strongest peak in the resulting data is found. This peak corresponds to water. The frequency where this peak is located is used as a reference, and it is centered at 0 Hz (or at the sampling frequency). Its value in terms of part per million (ppm) is assigned a value of 4.7 ppm. There are various ways for removing the water, e.g., applying finite impulse response (FIR) filtering (as described in J. P. Poullet, D. M. Sima, and S. Vab Huffel, “An automated quantitation of short echo time MRS spectra in an open source software environment: AQSES,” NMR in Biomedicine, vol. 20, 493-504, 2007) or using singular value decomposition of the data arranged in a Hankel matrix (HSVD) (as described in L. Vanhamme, R. D. Fierro, S. Van Huffel, and R. de Beer, “Fast removal of residual water in proton spectra,” Journal of Magnetic Resonance, vol. 132, 197-203, 1998). When HSVD is applied, the signal poles corresponding to the water are computed and from them the signal frequencies and damping factors of the water are estimated. The amplitudes of the signals are estimated by a least squares method and the water signal is constructed from the estimated parameters. Finally, the water signal is subtracted from the raw data. The residual is a signal with only a small water component.

At stage 214, an inquiry is made as to whether the water removal is successfully carried out. The spectrum of the water-removed data is tested to verify whether this spectrum fits an expected MRS spectrum. The fitness of the obtained spectrum is decided based on a predefined distance metric that measures how different the obtained spectrum is from the expected spectrum. The spectrum after water removal should not have a varying baseline and should have all the peaks of the strong metabolites. If the water is removed adequately, then the process 210 proceeds to stage 216, and otherwise returns to stage 212. The computer 116 preferably will determine that the water removal is not adequate a limited number of times. If this limit is reached, then the process 210 will end instead of returning to stage 212.

At stages 216-218, a signal-to-noise (SNR) ratio is estimated and compared against a threshold. Here, for example, an HSVD method is used with a predetermined order to estimate the SNR. The computer 116 finds the strengths of the signals that correspond to the main metabolites and estimates the noise in the data. For the signal estimate, the computer 116 uses the signal strength of a stable metabolite (usually Creatine). The computer 116 preferably computes the power of the signal from the estimated initial amplitude of the signal and its damping factor. If the signal's amplitude is A, the signal's damping factor is a, and the number of samples is N, then the total power of the signal is computed by

$$P = \frac{{\vert A \vert}^{2} ( 1 - {e}^{- 2 α N} )}{1 - {e}^{- α N}} .$$

The noise estimate may be obtained from part of the spectrum that contains no metabolites. At stage 218, the SNR is compared to a threshold, and if the SNR is below the predefined threshold, then the process 210 proceeds to stage 219 where the process 210 stops and it is declared that the data are not of sufficient quality. If the SNR is above the threshold, then the process 210 continues to stage 220.

At stage 220, the computer 116 performs line broadening on the data. The computer 116 multiplies the water-removed data by an exponential function. This smoothes the data and may improve the performance of the HSVD method.

At stage 222, the computer calibrates the data. This stage helps ensure that the frequencies of the metabolites appear where they should. The calibration is based on identifying known metabolites, for example, N-acetylaspartate (NAA), or lactate doublets. The selection of the metabolite for calibration depends on the type of data that are being processed. The NAA signal should be at 2.02 ppm and the lactate doublets at 1.33 ppm. The calibration is carried out by identifying the metabolites and computing the difference between their frequencies obtained from the data and their expected frequencies. The data are modulated with a complex sinusoid whose frequency is equal to the computed difference. The calibration may be implemented as multiple subprocesses.

At stage 224, the computer applies a passband filter. The band of interest is typically from 0 ppm to 4 ppm. The computer extracts the data for this band, and discards the remainder of the data. The filtering can be applied, e.g., by an FFT-based method.

At stage 226, data corresponding to frequencies nearby to a desired metabolite frequency are removed. Strong signals nearby the desired signal may affect the data for the desired signal, and are thus preferably removed. The computer 116 processes the data, e.g., using the HSVD method by forming the Hankel matrix, computing the SVD of the matrix, and constructing signals from the estimated signal parameters. The computer 116 removes these signals from the data. As with the water data removal, less than all of the data of these signals may in fact be removed.

At stage 228, the computer applies another passband filter, narrower than the one applied in stage 224. This additional filtering of the data extracts a much narrower band around the desired value, here 1.28 ppm, than the filtering in stage 224. The bandwidth of the filtering depends on the type of data that are analyzed. The implementation of this step may be analogous to that of stage 224.

At stage 230, the computer 116 estimates signal components and reconstructs a spectrum from each set of estimated components. The data from stage 228 are analyzed for the presence of a signal at frequency 1.28 ppm. If HSVD is applied, the computer can start with a low order and estimate the signal components in the data. From the estimated signal parameters, i.e., the signal amplitudes, frequencies and damping factors, the computer 116 reconstructs the spectrum of the data and compares it with the spectrum obtained by FFT. For example, if the reconstructed spectrum is denoted by Sᵣ(ƒ) and the FFT spectrum by S(ƒ), where ƒ denotes frequency, then one possible way of determining if the two spectra are “compatible” is by computing max|Sᵣ(ƒ−S(ƒ)|. The computer 116 increases the order of the HSVD, e.g., by one, and estimates the signal components and computes the corresponding spectrum. The computer 116 repeats this process until a predefined number of different HSVD orders are completed. The highest order of the HSVD applied by the computer 116 may depend on the type of data that are analyzed.

At stage 232, the computer 116 stores desirable estimates. The computer 116 determines a difference between the estimated and FFT spectra. If the difference is smaller than a predefined threshold, then the computer 116 stores the results of the HSVD. Otherwise, the computer 116 discards the results.

At stage 234, an inquiry is made as to whether a signal is present for each of the estimates stored at stage 232. The computer 116 analyzes the results stored at stage 232 to decide if there is a signal at the desired location, in this example, 1.28 ppm. The computer 116 determines in how many of the iterations a signal was found in the interval [1.28−ƒ, 1.28+ƒ], where ƒ is some small frequency, typically of the order of 10⁻² ppm. If a signal was found, then the process 210 proceeds to stage 236, and otherwise proceeds to stage 238.

At stage 236, the computer 116 checks the damping factor for the signal determined to be present at stage 234. The computer 116 checks the damping factor to determine if the damping factor is in an acceptable range. This process may help reduce the possibility of accepting as a signal a component that represents noise. The range for the acceptable damping factor may be machine dependent and is predetermined for the type of data being analyzed. If line broadening was applied, the computer 116 corrects the estimated damping factor by an amount that was added during the line broadening. The process 210 proceeds to stage 238.

At stage 238, an inquiry is made as to whether all of the stored estimates have been checked for the presence of a signal. If not all estimates have been checked, then the process 210 returns to stage 234. Otherwise, the process 210 proceeds to stage 240.

At stage 240, an inquiry is made as to whether a signal with an acceptable damping factor has been found a sufficient number of times. If not, then the process 210 proceeds to stage 241 where the process 210 ends. If so, then the process 210 proceeds to stage 242.

At stage 242, the computer 116 compares the estimates for consistency. The computer 116 compares the variability of the estimated amplitudes with a threshold, although other techniques may be used. If the computed variance is smaller than a predefined threshold, then the process 210 proceeds to stage 244, and otherwise returns to stage 228.

At stage 244, the presence of a signal is declared. The computer 116 computes an estimate of the “actual” signal from the individual estimates. For example, the computer 116 can compute a combination of the estimates such as the average value of the obtained estimates, a weighted estimate, or another combination of the estimates. From the obtained estimated amplitude and damping factor, the computer 116 estimates the power of the signal (as in stage 216) and from it the relative quantity of the cells that contribute to the 1.28 ppm signal. The latter estimate is based on the correlation that exists between the strength of the signal (its power) and the quantity of the stem cells.

The disclosure also provides methods for using the 1.28 ppm biomarker and a signal enhancing algorithm to monitor NPC within the mammalian brain. These methods may be used to determine the need of a patient for NPC augmentation or ablation and the relevance of NPC to brain trauma and/or neurological and psychiatric disorders. The methods may also be used to monitor transplanted NPC to patients in need thereof and to monitor neurogenesis in a wide range of human neurological and psychiatric disorders and diseases, and to evaluate the efficacy of therapeutic interventions (NPC may also be injected intravenously).

Non-limiting examples of diseases and conditions that may be monitored by the methods described herein include, but are not limited to, neurodegenerative disorders and neural disease, such as dementias (e.g., senile dementia, memory disturbances/memory loss, dementias caused by neurodegenerative disorders (e.g., Alzheimer's, Parkinson's disease, Parkinson's disorders, Huntington's disease (Huntington's Chorea), Lou Gehrig's disease, multiple sclerosis, Pick's disease, Parkinsonism dementia syndrome), progressive subcortical gliosis, progressive supranuclear palsy, thalamic degeneration syndrome, hereditary aphasia, amyotrophic lateral sclerosis, Shy-Drager syndrome, and Lewy body disease; vascular conditions (e.g., infarcts, hemorrhage, cardiac disorders); mixed vascular and Alzheimer's; bacterial meningitis; Creutzfeld-Jacob Disease; and Cushing's disease).

The disclosed embodiments also provide for methods of monitoring a nervous system disorder related to neural damage, cellular degeneration, a psychiatric condition, cellular (neurological) trauma and/or injury (e.g., subdural hematoma or traumatic brain injury), toxic chemicals (e.g., heavy metals, alcohol, some medications), CNS hypoxia, or other neurologically related conditions. In practice, the disclosed compositions and methods may be applied to a subject or patient afflicted with, or diagnosed with, one or more central or peripheral nervous system disorders in any combination. Diagnosis may be performed by a skilled person in the applicable fields using known and routine methodologies which identify and/or distinguish these nervous system disorders from other conditions.

Non-limiting examples of nervous system disorders related to cellular degeneration include neurodegenerative disorders, neural stem cell disorders, neural progenitor cell disorders, degenerative diseases of the retina, and ischemic disorders. In some embodiments, an ischemic disorder comprises an insufficiency, or lack, of oxygen or angiogenesis, and non-limiting example include spinal ischemia, ischemic stroke, cerebral infarction, multi-infarct dementia. While these conditions may be present individually in a subject or patient, the disclosed methods also provide for the treatment of a subject or patient afflicted with, or diagnosed with, more than one of these conditions in any combination.

Non-limiting embodiments of nervous system disorders related to a psychiatric condition include neuropsychiatric disorders and affective disorders. As used herein, an affective disorder refers to a disorder of mood such as, but not limited to, depression, post-traumatic stress disorder (PTSD), hypomania, panic attacks, excessive elation, bipolar depression, bipolar disorder (manic-depression), and seasonal mood (or affective) disorder. Other non-limiting embodiments include schizophrenia and other psychoses, lissencephaly syndrome, anxiety syndromes, anxiety disorders, phobias, stress and related syndromes (e.g., panic disorder, phobias, adjustment disorders, migraines), cognitive function disorders, aggression, drug and alcohol abuse, drug addiction, and drug-induced neurological damage, obsessive compulsive behavior syndromes, borderline personality disorder, non-senile dementia, post-pain depression, post-partum depression, and cerebral palsy.

Examples of nervous system disorders related to cellular or tissue trauma and/or injury include, but are not limited to, neurological traumas and injuries, surgery related trauma and/or injury, retinal injury and trauma, injury related to epilepsy, cord injury, spinal cord injury, brain injury, brain surgery, trauma related brain injury, trauma related to spinal cord injury, brain injury related to cancer treatment, spinal cord injury related to cancer treatment, brain injury related to infection, brain injury related to US 2007/0270449 AI (Nov. 22, 2007) inflammation, spinal cord injury related to infection, spinal cord injury related to inflammation, brain injury related to environmental toxin, and spinal cord injury related to environmental toxin.

Non-limiting examples of nervous system disorders related to other neurologically related conditions include learning disorders, memory disorders, age-associated memory impairment (AAMI) or age-related memory loss, autism, learning or attention deficit disorders (ADD or attention deficit hyperactivity disorder, ADHD), narcolepsy, sleep disorders and sleep deprivation (e.g., insomnia, chronic fatigue syndrome), cognitive disorders, epilepsy, injury related to epilepsy, and temporal lobe epilepsy.

Other non-limiting examples of diseases and conditions that may be monitored by the methods described herein include, but are not limited to, hormonal changes (e.g., depression and other mood disorders associated with puberty, pregnancy, or aging (e.g., menopause)); and lack of exercise (e.g., depression or other mental disorders in elderly, paralyzed, or physically handicapped patients); infections (e.g., HIV); genetic abnormalities (down syndrome); metabolic abnormalities (e.g., vitamin B12 or folate deficiency); hydrocephalus; memory loss separate from dementia, including mild cognitive impairment (MCI), age-related cognitive decline, and memory loss resulting from the use of general anesthetics, chemotherapy, radiation treatment, post-surgical trauma, or therapeutic intervention; and diseases of the of the peripheral nervous system (PNS), including but not limited to, PNS neuropathies (e.g., vascular neuropathies, diabetic neuropathies, amyloid neuropathies, and the like), neuralgias, neoplasms, myelin-related diseases, etc.

Other conditions that can be beneficially monitored by increasing neurogenesis are known in the art (see e.g., U.S. Publication Nos. 20020106731, 2005/0009742 and 2005/0009847, 20050032702, 2005/0031538, 2005/0004046, 2004/0254152, 2004/0229291, and 2004/0185429, herein incorporated by reference in their entirety).

The disclosure provides a spectroscopic biomarker of NPC, as well as methodology to detect this biomarker in the live brain for identifying NPC. The NPC biomarker is readily detected in vitro using ¹H-NMR, and a new methodology is developed to detect the biomarker at low concentrations in the live brain using ¹H-MRS. SVD-based signal processing proved to be superior to the traditionally used Fourier transform and can be applied to a variety of imaging settings where low levels of a particular metabolite preclude its reliable detection in vivo.

### EXAMPLES

Materials and Methods: Cell Culture

Preparation of neurospheres: Neurosphere cultures were prepared essentially as described previously (Mignone et al., 2004). Embryonic day 12 (E12) brains of C57B1/6 mice were isolated and digested in 2 mg/mL collagenase type-2 for 2 hrs at 37° C. Cells were filtered through a 40 μm filter three times and plated at a density of 50,000 cells/mL on plates coated with 2-hydroxyethyl methacrylate. Cells were grown in Neurocult Basal Media (NBM) with 10% Proliferation supplement. Growth factors (EGF, FGF-2, 20 ng/mL) were added every two days (Mignone et al., 2004). Neurospheres were collected after 14 days and trypsinized to single cells. After washing in phosphate-buffered saline (PBS, pH 7.25), they were resuspended in PBS and analyzed at different concentrations (0.1-10×10⁶ cells per sample) using ¹H-NMR. For differentiation experiments, neurospheres were plated onto polyornithine/laminin-coated cover slips, and maintained in the NBM with 10% Differentiation supplement (Mignone et al., 2004).

Preparation of hair follicle derived spheres: The entire skin from the dorsum of a postnatal day 30 (P30) C57B1/6 mouse was excised. The skin was digested in trypsin for 1 hr at 30° C. and then 2 mg/mL collagenase type-2 for 2 hrs at 37° C. Cells were filtered three times using a 40 μm filter and then plated at 30,000 cells/mL on plates coated with 2-hydroxyethyl methacrylate. Cells were maintained in the NBM containing 10% Proliferation supplement for 2 weeks. Growth factors EGF and FGF-2 were added every two days (Mignone et al., 2007). The spheres were collected after 14 days and prepared for spectroscopy as above. Primary cultures of astrocytes: Astrocytes were derived from the P2 cortices of C57B16 mice, and digested in 2 mg/mL collagenase for 2 hrs at 37° C. (Maletic-Savatic et al., 1995). Cells were filtered three times using 40 μm filter and plated at 500,000 cells per 10 cm tissue culture dish coated with poly-D-lysine. Cells were maintained in Earle's MEM containing 10% horse serum and 0.6% glucose. The media was changed every two days. After reaching confluency (2 weeks), the cells were detached with trypsin/EDTA, washed three times, resuspended in the PBS, and analyzed at different concentrations (1-10×10⁶ cells per sample) by ¹H-NMR.

Primary cultures of neurons: Rat primary hippocampal neurons were purchased from QBM Cell Science. Neurons were plated on poly-D-lysine and laminin coated dishes and cultured in Neurobasal medium at a density of 200,000 cells/mL. The media was changed every 2 days. Two weeks after plating, neurons were collected, washed three times, and resuspended in the PBS for analysis by ¹H-NMR.

Primary cultures of oligodendrocytes: Primary oligodendrocytes were derived from the P2 cortices of C57B1/6 mice using a shaking method, as described (McCarthy and De Villis, 1980). Cultures were maintained in poly-D-lysine-coated 75 cm flasks in plating media (Dulbecco's modified Eagle's medium (DMEM), 20% fetal bovine serum, 1% penicillin-streptomycin) which was changed every 2 days. After 10 days, the flasks were shaken for 1 hr at 200 rpm to remove adherent microglia/macrophages, washed with the same medium, and then shaken overnight at 200 rpm to separate oligodendrocytes from the astrocyte layer. The suspension was plated onto uncoated Petri dishes and incubated for 1 hr at 37° C. to further remove residual microglia and astrocytes that adhere to the dishes. The oligodendrocytes were collected through a 15 μm sieve and plated onto poly-ornithine coated culture plates. Purified oligodendrocytes were cultured for 7-9 days in DMEM containing 0.1% bovine serum albumin, 50 μg/mL apo-transferrin, 50 μg/mL insulin, 30 nM sodium selenite, 10 nM D-biotin, 10 nM hydrocortisone, 200 μM L-cystine, 10 ng/mL PDGF, and 10 ng/mL basic FGF. After 2 weeks in culture, oligodendrocytes were collected and prepared for the ¹H-NMR as above.

Preparation of oligodendrocyte progenitor cells (OPC): Dissociated neonatal rat forebrains were cultured in DMEM with 10% fetal calf serum on poly-D-lysine coated flasks. After 2 weeks, the flasks were placed on a rotary shaker at 200 rpm for 1 hr to remove the majority of loosely adherent microglia. A subsequent prolonged shake for 16 hrs was used to dislodge OPC from the astrocyte monolayer into the supernatant. OPC were maintained in the Sato's media and processed immediately (Young and Levinson, 1997).

Other cell lines:

The Macrophage cell line (J774) (courtesy of Rebecca Rowehl, Stony Brook University) was maintained in DMEM supplemented with 10% FBS at 37° C.

The T cell line (Jurkat) (courtesy of Dr. Martha Furie, Stony Brook University) was maintained in RPMI supplemented with 10% FBS at 37° C.

Microglia were isolated from cultures of mixed cortical cells as described previously (Hassan et al., 1991). Briefly, cortices from the neonatal C57B1/6 mice were trypsinized and triturated, and the resulting single-cell suspension was plated into poly-L-Lysine coated 75 cm₂ tissue culture flasks. The medium (DMEM, 10% FBS, and 40 mg/L gentamycin) was changed every 3 days. After 10-14 days in culture, the mixed cortical cells establish a confluent layer with bright rounded microglial cells visible on top of the layer. These microglia were removed by 15 mM lidocaine treatment with gentle shaking. After centrifugation, the pellet was resuspended in the medium and plated onto poly-L-Lysine coated glass coverslips.

Isolated embryonic stem cells (ESC, courtesy of Dr. Alea Mills, Cold Spring Harbor Laboratory) were seeded in a 35 mm tissue culture dish containing a confluent layer of mouse embryonic fibroblasts. ESC were grown at 37° C. in DMEM containing 15% FCS, 1% L-glutamine, 1% Non-essential amino acids, 1% Penicillin/Streptomycin, 0.2% mercaptoethanol and 0.0001% leukemia inhibiting factor. ESC were passaged every 2-3 days.

Single cell suspensions isolated from mouse whole brains. Whole brains, isolated from E12 and adult P30 C57B1/6 mice, were digested in 2 mg/ml collagenase type-2 for 2 hrs at 37° C. Cells were filtered through a 40 μm filter three times and washed in PBS prior to ¹H-NMR analysis.

Biochemical experiments. Neurospheres were treated with cerulenin (5 μg/mL) for 24 hrs at 37° C. They were then trypsinized, washed, and resuspended in PBS for ¹H-NMR. To test if the 1.28 ppm biomarker metabolite is in the lipid fraction, neurospheres were solubilized in chloroform:methanol (2:1), sonicated, and analyzed by ¹H-NMR. ¹H-NMR was also done for palmitic acid (SFA), oleic acid (MUFA), and arachidonic acid (PUFA) as controls, used at 100 μg/mL.

Gas Chromatography (GC). GC was performed by Scientific Research Consortium, Inc. (SRC, St Paul, Minn.). Lipids from NPC and astrocytes were extracted with chloroform:methanol 2:1, the sample was mixed and centrifuged, the aqueous layer was decanted, and the chloroform layer filtered to remove the protein. The solution was then dried with a stream of nitrogen and redissolved in chloroform. The lipid extracts were then applied to silica gel thin-layer plates and developed in petroleum ether/diethyl ether/acetic acid, 80:20:1. Lipid classes were visualized 0.1% 2,7-dichlorofluorescein solution under UV light, and then scraped into glass tubes with teflon screw caps. The free fatty acid class was trans esterified with 12% BF3 in methanol at 75° C. The resulting methyl esters were extracted with water and petroleum ether, dried under nitrogen, re-dissolved in heptanes and analyzed by GC. GC analysis was carried out with a gas chromatograph equipped with a 50 m×0.25 mm capillary column and linked to an integrator. The column was temperature programmed from 180-220° C. at 2° C./min with an initial time of 10 min and a final time of 30 min. Helium carrier gas and a split ratio of 100:1 was used. Identification of fatty acid peaks was made by comparison with authenticated standards.

NPC transplantation. Five million NPC (1×10⁶ cells/μL) were grown in vitro as neurospheres and trypsinized to single cells before transplantation. NPC were injected transcranially into the left hemisphere of an adult rat cortex (stereotaxic coordinates X:Y:Z=1:1:3 mm from the bregma). The same volume of PBS (5 μL) was injected into the contralateral hemisphere as a sham control. Rats were imaged within 4 hrs after injection, as described below. All animal care was in accordance with institutional guidelines.

Electroconvulsive shock (ECS). ECS experiments were performed using a Ugo Basile (57800) ECS unit. Bilateral ECS was administered via moistened pads on ear clips using a pulse generator in male adult C57B1/6 mice (frequency 50 Hz, shock duration 0.5 msec, pulse width 0.5 msec, and current 50 mA) and in male adult Sprague Dawley rats (frequency 100 Hz, shock duration 0.5 msec, pulse width 0.5 msec, and current 50 mA) (Sartorius et al., (2003). ECS was performed at the same time each day for five consecutive days. On the fifth day, animals were injected with 150 mg/kg BrdU. Sham-control animals were exposed to the same procedure, but did not receive the shock. Twenty-four hours later, mice where either sacrificed and their hippocampi dissected and prepared for ¹H-NMR, or they were perfused and brains prepared for immunostaining with the anti-BrdU antibody. Rats were imaged by mMRI spectroscopy as outlined below. Following mMRI, they were perfused, brains were fixed, sectioned, and immunostained with the anti-BrdU antibody.

NMR spectroscopy. One-dimensional ¹H-NMR spectra of the aqueous suspensions of cells (0.55 mL, pH 7.25) containing 10% D₂O as a field frequency lock were measured using a 700 NMR spectrometer. Spectral analysis was conducted using XWinNMR, version 3.5. In all the experiments, the temperature was maintained at 35° C. and a pH of 7.25. The spectra were acquired with a Free Induction Decay (FID, 32,768 points in a spectral width of 8389.3 Hz, readout time of 1.95 seconds, repetition time of 2 seconds and 128 averages). The water signal was pre-saturated with a low power radiofrequency (RF) pulse. Before Fourier transform, FIDs were line broadened to 1.0 Hz with an exponential weighting function. ¹H-NMR spectra were phase and baseline corrected for distortions.

MicroMRI acquisition. Proton mMRI spectroscopy was performed on a 9.4T Biospec Avance 94/20 as scanner. Adult Sprague-Dawley rats were anesthetized with ketamine/xylazine mixture and allowed to breathe spontaneously. Anesthesia was maintained with 1-1.5% isoflurane in a 1:10₂/air mixture delivered via a T-piece. The rat head was immobilized in a custom-built head holder and positioned on a 3.0 cm RF surface coil. Initial hydration was administered by an intraperitoneal injection of Lactated Ringer (4 mL/kg/hr). Fluids were continuously administered through the intraperitoneal catheter. To reduce salivation and maintain optimal conditions for spontaneous ventilation, glycopyrulate (0.1 mg/kg) was given prior to positioning within the mMRI scanner. The rat ears were covered with cotton/gauze custom-build ear fittings to protect against noise from the mMRI gradients during scanning. A high resolution axial T2 image was used to specify the spectroscopy voxel of interest (VOI, 2.5 mm³), located within the hippocampus for imaging of endogenous NPC and within the cortex for imaging of transplanted NPC. Spectra were collected using point resolved spectroscopy (PRESS), TE/TR=8 ms/2,000 ms, and 2,048 data points extending over a spectral width of 16.01 ppm (6410 Hz), yielding a spectral resolution of 1.57 Hz/point (imaging time 54 min). The suppression bandwidth was a 400 Hz CHESS (chemical-shift selective) pulse. First and second order shims were accomplished using the Fastmap sequence followed by automatic local shim adjustment. The spectrometer frequency and receiver gain were adjusted to achieve 50-60% digitizer filling. Pulse angles were further adjusted to reduce the digitizer filling to between 30-45%. All animal care was in accordance with institutional guidelines.

Human brain MRI. Adult human brain ¹H-MRS was obtained using a 3T MRI scanner, with the following parameters: TE/TR=14 ms/2,000 ms, voxel size 30×12×12 mm³ oriented along the hippocampus and 16×16×16 mm³ within the gray matter in the cortex, spectral width 2,000 Hz, 1,024 points, 128 averages, and total image time 4 min 55 sec. Five healthy volunteer subjects of both sexes were tested at baseline and 90 days after. In addition, three pre-adolescent (8-10 year old) and three adolescent (14-16 year old) were imaged using the same parameters. Spectra of cortical and hippocampal regions were obtained and analyzed with custom-made SVD-based signal processing. Informed consent was obtained from all volunteers.

SVD-based signal processing. The signal processing method is implemented interactively and is based on a parametric approach. It is assumed that the data can be represented by a Lorentzian model, which is a superposition of decayed complex sinusoids. Each sinusoid is identified by four parameters, amplitude, initial phase, frequency, and damping factor of which the frequency and the damping factor are nonlinear parameters. The various resonances are due to the different metabolites in the sample, and their intensities are proportional to the number of nuclei that resonate at the corresponding frequencies. The method proceeds as follows. First, a Fast Fourier transform (FFT) of the raw data is computed with the objective of finding the strongest peak in the spectrum of the data. This peak corresponds to water, and the frequency where it is located is used as a reference and is centered at 0 Hz (or at the sampling frequency). Its value in terms of part per million (ppm) is assigned as 4.7 ppm. In the next step the water is removed from the data. The HSVD method (singular value decomposition of the acquired signal arranged in a Hankel matrix) is applied by first computing the signal poles that correspond to water and from them the signal frequencies and damping factors (Barkhusen et al., 1987). The poles corresponding to water are identified by their location in the z-plane. Then the amplitudes of the signal components are estimated by the least squares method, and the water signal is constructed from the estimated parameters. Subsequently, the water signal is subtracted from the raw data. The spectrum of the signal after water removal is checked for presence of remaining water. If additional water removal is desired, the process is repeated on the residual data. After water removal, the obtained data is multiplied with a decaying exponential function with the purpose of increasing the overall SNR. The function is of the form e^{-2πctsn}, where n=0,1,2, . . ., N−1 with N being the length of the data set, tₛ is the machine's sampling interval, and c is a user defined constant (for in vitro, human, and the rat data used c=0.5, 0.7, and 3, respectively).

Next, a second frequency alignment is performed. An FFT is applied to the resulting time series data from the previous step. In the obtained spectrum, one can clearly see either the lactate doublets for the in vitro samples, or the NAA peak for the in vivo samples. The frequency alignment is performed by centering the lactate doublets to 1.33 ppm, or by adjusting the position of the NAA peak to 2.02 ppm.

The frequency band of interest is filtered. The ER (extraction and reduction) filter is applied from the filtering amounts to apply FFT to the water-removed data followed by selecting the frequency bandwidth of interest (Cassila et al., 1997). Then, the selected spectrum is shifted to the baseband and the inverse FFT is applied. In the time domain, these operations correspond to convolution of the water-removed signal with the impulse response of an ideal bandpass filter, modulation of the filtered signal, and decimation with a factor equal to the ratio of the original bandwidth and the bandwidth of interest. The filtered data are then again processed using the HSVD method by first forming the Hankel matrix and computing the SVD of the matrix and the modes of the signal. From the obtained modes, the corresponding amplitudes are found. With the obtained parameters, signals are constructed that are desired to be removed from the filtered data (these signals are the ones close to the NPC signal in the frequency domain). This procedure is in general conducted iteratively. In the first iteration, the stronger signal components are estimated and removed, and in the following iterations, the weaker ones. Typically, however, the removal of the signals after the first iteration is sufficient. Once the filtering is completed, there is an additional fine-tuning of the frequency, which is followed by detection of the NPC peak. If there is a mode in the final set of data within the window of 1.28±0.025 ppm, then it is declared that there are NPC in the sample, and from the peak area of the mode, their quantity is estimated. Other embodiments of methods, including methods with different orders than that described above, may be used and are within the scope of the disclosure and claims.

Statistics. Statistical analysis was performed using Statistica. In experiments comparing two groups, Student t-test was used. In experiments comparing three or more groups, the normal distribution of the data histograms and the homogeneity of variances (Levene's test) were determined before Analysis of Variance (ANOVA). ANOVA was followed by post-hoc analysis with Tukey-HSD test (High Significance Difference) for pair-wise multiple comparisons or with the Dunnet test when comparing experimental with a control group. In linear regression analysis, the correlation coefficient R₂ is indicated. All quantification was done with the SVD-based method. Bar graphs represent mean +/− SEM; *, p<0.05; **, p<0.01; and ***, p<0.001. Detailed statistics data for each figure are listed in Supporting Text.

### SUPPORTING TEXT

FIG. 1. The 1.28 ppm biomarker identifies Neural Progenitor Cells. (B) The 1.28 ppm mean peak areas are: NPC: 1,870±214; N: 4.10±0.32; O: 338.0±82.9; and A: 7.10±0.41.

Statistics: ANOVA p=0.000007, Dunnet test: NPC vs N p=0.000016, NPC vs O p=0.00004, and NPC vs A p=0.000016. NAA mean peak areas are: NPC: 6.9±3.5; N: 49.4±5.2; O: 6.20±0.66; and A: 11.2±2.8. Statistics: ANOVA p=0.000047, Dunnet test: N vs NPC p=0.000064, N vs O p=0.000059 and N vs A p=0.00013. Cho mean peak areas are: NPC: 74.1±7.1; N: 169.1±18.9; O: 98.7±7.8; and A: 274.0±8.6. Statistics: ANOVA p=0.000007, Dunnet test: A vs NPC p=0.000014, A vs N p=0.00052, and A vs O p=0.000022. (C) The 1.28 ppm mean peak areas are: NPC: 15,700±1,130; ESC: 1,370±177; SSC: 6,470±1,530; OPC: 5,530+823; MΦ: 237±96; TC: 100±23; and MG: 657±146. Statistics: ANOVA p<0.000001, Dunnet test: NPC vs ESC p=0.000009, NPC vs SPC p=0.000014, NPC vs OPC p=0.00001, NPC vs MΦp=0.000009, NPC vs TC p=0.000009, and NPC vs MG p=0.000009. (D) The 1,28 ppm mean peak areas are: 0.25×10⁶ NPC=5,050±435; 0.5×10⁶ NPC=8,810±669; and 1×10⁶ NPC=16,500±721.

FIG. 2. Analysis of the specificity and molecular composition of the NPC biomarker using ¹H-NMR. (A) The 1.28 ppm mean peak areas are: D0: 12,400±324; D1: 13,600±392; and D5: 3,890±158. Statistics: ANOVA p=0.000001, Tukey test: DO vs D1 p=0.084, DO vs D5 £=0.00023 and D1 vs D5 £=0.00023. The NAA mean peak areas are: DO: 1,220±219; D1: 2,040+37; D5: 2,100±37. Statistics: ANOVA £=0.0046, Tukey test: DO vs D1 p=0.0093, DO vs D5 p=0.0066 and D1 vs D5 p=0.94. The Cho mean peak areas are: DO: 74±7; D1: 156±14 and D5: 351±12.7. Statistics: t-test, p<0.000001. (B) The 1.28 ppm mean peak areas are: E12: 3,080±244; P30: 450±43, t-test p<0.000001. The NAA mean peak areas are: E12: 4,220±853; P30: 16,600±1,470; t-test p=0.002. The mI mean peak areas are: E12: 300±7; P30: 430±31; t-test p=0.013. (C) The 1.28 ppm mean peak areas are: CTX: 276±76; and HIPP: 1,390±205; t-test p=0.007. (E) The 1.28 ppm mean peak areas are: control (CTRL): 12,600±665; CRL: 5,570+751, t-test p=0.00078. The Cho mean peak area are: CTRL: 310±8; CRL: 443+6, t-test p=0.00077.

FIG. 3. Identification of NPC in the rat brain in vivo, using microMRI spectroscopy. (A). The 1.28 ppm mean peak areas are: CTX: 2,790±966; and HIPP: 32,100±8,480; t-test p=0.026. 1.28 ppm:Cr ratios are: CTX: 0.00814±0.00140; and HIPP: 0.100±0.038; t-test p=0.076. (B) The 1.28 ppm mean peak areas are: ST: 12,000±1,210; and NT: 417,000±123,000; t-test p=0.029. 1.28 ppm:Cr ratios are: ST: 0.037±0.037; and NT: 0.817±0.220; t-test p=0.025. (C) The 1.28 ppm:Cr ratios are: ECS−: 0.0312+0.0070; and ECS+: 0.0647±0.0113; t-test p=0.046. (D) The number of BrdU positive cells: ECS−: 975+93; and ECS+: 1,670±166; t-test p=0.011.

FIG. 4. Identification of NPC in the human hippocampus in vivo, using 'H-MRI spectroscopy. (A) The 2.28ppm mean peak areas are: CTX: 0.116×10⁻⁶±0.126×10⁻⁶; left hippocampus (LH): 2.47×10⁻⁶±0.78×10⁻⁶; and right hippocampus (RH): 1.88×10⁻⁶±0.89×10⁻⁶. Statistics: ANOVA p=0.004, Dunnet test: CTX vs LH p=0.0028 and CTX vs RH p=0.021. The 1.28 ppm:Cr ratios are: CTX: 0.217×10⁻⁶±0.230×10⁻⁶ LH: 3.12×10′₂±0.86×10⁻⁶; and RH: 2.42×10⁻⁶+0.74×10⁻⁶. Statistics: ANOVA p=0.023, Dunnet test: CTX vs LH p=0.016 and CTX vs RH p=0.069. (B) The 1.28 ppm peak areas are: Day 1: 2.47×10⁻⁶±0.78×10⁻⁶, t-test p=0.747; Day 90: 3.13×10⁻⁶±1.05×10⁻⁶. (C) The 1.28 ppm mean peak areas are: pre-adolescent: 50.0×10⁻⁶±4×10⁻⁶, adolescent: 26×10⁻⁶±3×10⁻⁶ and adult: 4×10⁻⁶±14×10⁻⁶. Statistics: ANOVA p<0.000001, Dunnet test: pre-adolescent vs adolescents p=0.004, pre-adolescent vs adult p<0.000001 and adolescent vs adult p<0.000001.

Other embodiments are within the scope and spirit of the appended claims. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, more than one invention may be disclosed.

What is claimed is:

1. A Nuclear Magnetic Resonance (¹-H-NMR) and/or Magnetic Resonance Spectroscopy (¹H-MRS) spectral biomarker appearing as a peak in the (¹H-NMR and/or (¹H-MRS spectra in the area of approximately 1.28 (+/−0.02) ppm after water removal.

2. The biomarker of claim 1 wherein the biomarker has higher concentrations in the hippocampus and/or subventricular zone compared to other regions of a normal mammalian brain.

3. The biomarker of claim 1 wherein the biomarker comprises a mixture of lipids.

4. The biomarker of claim 3 wherein the mixture of lipids comprises saturated fatty acid (SFA) and/or monounsaturated fatty acids (MUFA).

5. A method of detecting neural stem and/or progenitor cells comprising the steps of:
a) scanning a sample of tissue or cells using Nuclear Magnetic Resonance (¹H-NMR) and/or Magnetic Resonance Spectroscopy (¹H-MRS); and
b) detecting a spectral biomarker in the area of approximately 1.28 (+/−0.02) ppm after water removal, wherein the biomarker indicates the presence of neural stem and/or progenitor cells.

6. The method of claim 5 wherein the neural stem and/or progenitor cells are detected in vivo.

7. The method of claim 5 further comprising the step of preparing the sample of tissue or cells for Nuclear Magnetic Resonance (¹H-NMR) and/or Magnetic Resonance Spectroscopy (¹H-MRS) analysis.

8. The method of claim 5 wherein the intensity (area under the curve) of the peak of the biomarker in the area of approximately 1.28 ppm is used to quantify the number of neural stem and/or progenitor cells within the sample.

9. The method of claim 5 wherein the sample of tissue or cells is in a selected area within a mammalian brain.

10. The method of claim 9 wherein the selected area is the hippocampus and/or the subventricular zone and/or the cortex of the mammalian brain.

11. A method of monitoring transplanted neural stem and/or progenitor cells in a selected area of a mammalian brain comprising the steps of:
a) scanning a sample of tissue or cells using Nuclear Magnetic Resonance (¹H-NMR) and/or Magnetic Resonance Spectroscopy (¹H-MRS); and
b) detecting a spectral biomarker in the area of approximately 1.28 (+/−0.02) ppm after water removal, wherein the biomarker indicates the presence of neural stem and/or progenitor cells.

12. A method of evaluating the efficacy of a therapeutic intervention as measured by an increases or decrease in the number of neural stem and/or progenitor cells in a selected area of a mammalian brain comprising the steps of:
a) scanning a sample of tissue or cells using Nuclear Magnetic Resonance (¹H-NMR) and/or Magnetic Resonance Spectroscopy (¹H-MRS); and
b) detecting a spectral biomarker in the area of approximately 1.28 (+/−0.02) ppm after water removal, wherein the biomarker indicates the presence of neural stem and/or progenitor cells.

13. The method of claim 12 wherein the therapeutic intervention is for a nervous system disorder related to cellular degeneration, a psychiatric condition, cellular trauma and/or injury, or another neurologically related condition in a mammalian subject or patient.

14. The method of claim 13 wherein the nervous system disorder related to cellular degeneration is a neurodegenerative disorder, a neural stem cell disorder, a neural progenitor cell disorder, a degenerative disease of the retina, an ischemic disorder, or combinations thereof.

15. The method of claim 13 wherein the nervous system disorder related to a psychiatric condition is a neuropsychiatric disorder, an affective disorder, depression, hypomania, panic attacks, anxiety, excessive elation, bipolar depression, bipolar disorder (manic-depression), seasonal mood (or affective) disorder, schizophrenia and other psychoses, lissencephaly syndrome, anxiety syndromes, anxiety disorders, phobias, stress and related syndromes, cognitive function disorders, aggression, drug and alcohol abuse, obsessive compulsive behavior syndromes, borderline personality disorder, non-senile dementia, post-pain depression, post-partum depression, cerebral palsy, post-traumatic distress disorder (PTSD), or combinations thereof.

16. The method of claim 13 wherein the nervous system disorder related to cellular trauma and/or injury are neurological traumas and injuries, surgery related trauma and/or injury, retinal injury and trauma, injury related to epilepsy, spinal cord injury, brain injury, brain surgery, trauma related brain injury, trauma related to spinal cord injury, brain injury related to cancer treatment, spinal cord injury related to cancer treatment, brain injury related to infection, brain injury related to inflammation, spinal cord injury related to infection, spinal cord injury related to inflammation, brain injury related to environmental toxin, spinal cord injury related to environmental toxin, or combinations thereof.

17. The method of claim 13 wherein the neurologically related condition are learning disorders, memory disorders, autism, attention deficit disorders, narcolepsy, sleep disorders, cognitive disorders, epilepsy, temporal lobe epilepsy, or combinations thereof.

18. The method of claim 15 wherein the psychiatric condition comprises depression.

19. The method of claim 15 wherein the psychiatric condition comprises PTSD.

20. The method of claim 12 wherein the increase or decrease in the number of neural stem and/or progenitor cells is affected through the therapeutic intervention.

21. The method of claim 20 wherein the increase in the number of neural stem and/or progenitor cells is correlated with increased neurogenesis, and wherein the decrease in the number of neural stem and/or progenitor cells is correlated with decreased neurogenesis.

22. The method of claim 12 wherein the mammalian brain is a human brain.

23. A signal-processing algorithm for isolating a ¹H-MRS signal from background noise.

24. The algorithm of claim 23 utilized to enhance the ¹H-MRS spectra.

25. A system for identifying the presence of a biomarker from MRS data, the system comprising:
an input configured to receive the MRS data;
a memory configured to store the MRS data; and
a processor configured to operate on the stored MRS data to:
reduce an influence of water data in the MRS data;
determine that a signal-to-noise ratio of the MRS data with reduced water data influence is within a desired range;
calibrate the MRS data with reduced water data influence;
reduce influences of signals in close proximity, as a function of parts per million (ppm), to a ppm value of interest; and
determine whether a signal exists approximately at the ppm value of interest

26. The system of claim 25 wherein the processor, in order to determine whether a signal of a strength above a threshold exists at the ppm value of interest, is configured to:
estimate sets of signal components using models of different orders;
construct spectra from the estimated sets of signal components; and
determine if the constructed spectra are within an acceptable difference relative to a calculated spectrum determined using a Fourier transform of the MRS data.

27. The system of claim 26 wherein the processor, in order to determine
whether a signal of a strength above a threshold exists at the ppm value of interest, is further configured to:
determine whether a sufficiently strong signal, of a strength above a threshold, exists in a vicinity of the ppm of interest for each of the estimates for which the constructed spectra was within the acceptable difference; and
determine whether a damping factor for each sufficiently strong signal is in an acceptable range.

28. The system of claim 27 wherein the processor, in order to determine
whether a signal of a strength above a threshold exists at the ppm value of interest, is further configured to compare the sufficiently strong signals for consistency.

29. The system of claim 28 wherein the processor, in order to determine
whether a signal of a strength above a threshold exists at the ppm value of interest, is further configured to combine the sufficiently strong signals into an indication of the signal that exists approximately at the ppm value of interest.

30. The system of claim 29 wherein the processor is configured to use the indication to estimate a quantity of cells.

31. A computer program product residing on a computer-readable medium comprising computer-readable instructions that will cause a computer to:
reduce an influence of water data in the MRS data;
determine that a signal-to-noise ratio of the MRS data with reduced water data influence is within a desired range;
calibrate the MRS data with reduced water data influence;
reduce influences of signals in close proximity, as a function of parts per million (ppm), to a ppm value of interest; and
determine whether a signal exists approximately at the ppm value of interest.

32. A computer program product residing on a computer-readable medium comprising computer-readable instructions that will cause a computer to:
perform analysis of metrics of parametric and non-parametric spectra of MRS data; and
determine from the analysis whether estimates of signals within the MRS data are acceptable.

33. A computer program product residing on a computer-readable medium comprising computer-readable instructions that will cause a computer to:
perform analysis of parametric and non-parametric spectra of MRS data; and
determine from the analysis whether a biomarker is indicated by the MRS data.

34. The computer program product of claim 33 wherein the instructions will
further cause the computer to determine a set of acceptable spectra and determine from the set of acceptable spectra whether the biomarker is indicated by the MRS data.

35. A computer program product residing on a computer-readable medium comprising computer-readable instructions that will cause a computer to:
estimate signal parameters, indicated by MRS data, for a biomarker of interest; and
estimate a quantity of tissue cells contributing to a signal strength of the biomarker.
